(19) United States
(12) Patent Application Publication
(10) Pub. No.: US 2017/0007439 A1
(43) Pub. Date: Jan. 12, 2017

(54) Ostomy Appliance Collar

(71) Applicant: Thomas J. Boksan, Bethlehem, PA (US)

(72) Inventor: Thomas J. Boksan, Bethlehem, PA (US)

(21) Appl. No.: 15/207,465

(22) Filed: Jul. 11, 2016

Related U.S. Application Data
(60) Provisional application No. 62/191,072, filed on Jul. 10, 2015.

(51) Int. Cl. A61F 5/445; A61F 5/448; A61F 5/443

(52) U.S. Cl. CPC A61F 5/445; A61F 5/443; A61F 5/448

(57) ABSTRACT

The present invention provides an accessory and/or manufacturing stage addition to current ostomy devices which is capable of remediating or preventing various causes for concerns for the myriad populations of patients who rely on such devices.

### CROSS REFERENCE TO RELATED APPLICATIONS

The present application claims priority under 35 U.S.C. §119(e) from U.S. Provisional Application Ser. No. 62/191,072, filed Jul. 10, 2015. The entire contents of this application are incorporated by reference herein.

### FIELD OF THE INVENTION

The present invention pertains most notably to the field of medical devices, specifically ostomy devices and accompaniments, and accessories therefor.

### BACKGROUND OF THE INVENTION

Ostomy surgery, also known as bowel diversion, refers to surgical procedures that entail the rerouting of the usual progress of intestinal contents out of the body, to functionally bypass a section of the bowel that is removed or diseased. Part of the intestine is positioned through the abdominal wall, in order to allow waste to exit a patient, through the abdominal wall into some synthetic receptacle, instead of passing through the anus. Ostomy surgeries are used as treatments for myriad conditions and their symptoms and the resulting stoma and other physical outcomes of such surgeries may be temporary or permanent remedies, depending on the etiology of the medical condition necessitating the surgery.

After all surgeries to implement, or result in the temporary or permanent need for, ostomy appliances, patients experience a trial period, during which each patient must identify how to best use his ostomy appliance and accessories and/or what additional accessories he may need for temporary or permanent use. There is huge variation in each patient's optimal ostomy care and operation, depending on body type and shape, skin texture and sensitivity, personal preferences and the nature of the stoma and effluent. During this often protracted and trying time, made more difficult by the patient's post-surgical status, improper ostomy appliance fit can cause physical discomfort, decreased adhesion duration times, blow-outs, leakage, and emotional distress.

According to Best Practice Guidelines for the Management of the patient with a fecal ostomy, each year approximately 100,000 people in the United States undergo surgical operations that result in a colostomy or ileostomy each year. (See Goldberg M, Aukett L K, Carmel J, et. al. J Wound Ostomy Continence Nurs. 2010; 37: 596-598.) Due to the variability among patients, with respect to underlying conditions, surgical circumstances, skin textures and sensitivities, and ostomy types and functions, the problems noted herein pose a serious problem for patients and care providers and pose a unique and unanswered challenge for device manufacturers. These problems—inconvenience, emotional stress, and disruption to a patient's recovery and acclimatization to his physical condition (whether permanent or temporary) caused by improper ostomy appliance fit, decreased adhesion duration times, and blow-outs or leakage—are resolved by the present invention.

### SUMMARY OF THE INVENTION

The present invention (the “ostomy appliance collar” or the “collar”) is a thin, soft, pliable, transparent, pressure-sensitive, adhesive, collar or surround used in conjunction with an ostomy appliance, made from plastic or other appropriate materials, which will prevent leakage and discharge from the patient's receptacle device. The present invention provides further benefit to the user by providing adequate and flush adhesion of the ostomy appliance adhesive or sealing wafer to the skin barrier, increasing the patient's comfort level and emotional composure and decreasing the breakdown of secure adhesion from uneven pressure exerted by the wafer and thus the likelihood of leakages.

The versatility and adaptability of the present invention will serve the long felt, but unmet, need of patients with stomas by accommodating various patient body types, medical needs of the patient and device-specific features and appliance variability. The present invention will be helpful to patients with all forms of urostomies, colostomies, and ileostomies and has applications within both one-piece and two-piece systems. Products may be modified by the end-user and/or customized by manufacturers to fit the various needs of each affected patient population.

### BRIEF DESCRIPTIONS OF THE FIGURES

Unless otherwise stated, all dimensions are in inches and drawings are not to scale.

FIGS. 1A-1D illustrate different views of the present invention: FIG. 1A shows an exploded view of the ostomy appliance collar; FIG. 1B is a proximal view of the ostomy appliance collar with the release liner intact; FIG. 1C shows the adhesive layer side of the backing layer of the ostomy appliance collar with the release liner removed; and FIG. 1D is the distal face of the ostomy appliance collar with the support layer attached.

FIGS. 2A-2D illustrate the method by which the user would prepare the ostomy appliance for correct placement of the present invention: FIG. 2A illustrates a folded ostomy appliance which would be passed through the present invention to the skin barrier/wafer; FIG. 2B illustrates the direction of movement for the pre-positioning of the present invention; FIG. 2C demonstrates the process by which the user would push/pull the edges of the ostomy appliance through the center portion of the present invention; and FIG. 2D illustrates a properly positioned ostomy appliance collar.

FIGS. 3A-3C indicate the timing and process for removal of the release liner from the present invention as displayed in FIG. 1B: FIG. 3A shows the left side of the release liner being removed; FIG. 3B shows the right side of the release liner being removed; and FIG. 3C illustrates how the user would gently strip the release liner from the adhesive layer and apply light pressure at first to seat the collar and then firmer pressure to secure the ostomy appliance collar to the skin-barrier/wafer and body.

FIGS. 4A and 4B explain the timing and process for removing the support layer as noted in FIG. 1D: FIG. 4A shows the invention as pictured in FIG. 1D with the support layer being removed; and FIG. 4B illustrates how to remove the support layer from the front face of the ostomy appliance collar.

FIG. 5 provides the iteration of the present invention with the perforated tab removed.

FIG. 6 illustrates the present invention, in use, with the ostomy appliance unfolding to wearing position.

FIG. 7 shows the transparent front face of the ostomy appliance collar with the support layer, release liner, and perforated tab removed.

### DETAILED DESCRIPTION OF INVENTION AND PREFERRED EMBODIMENTS

The present invention is a thin, soft, pliable, transparent, pressure-sensitive, adhesive, collar or surround used in conjunction with an ostomy appliance, made from plastic or other appropriate materials. The invention's outer circumference is specifically sized to complement the larger outer circumference of the skin-barrier/wafer of the ostomy appliance, subject to variation but which can be produced according to standard sizes and needs of patients, and this prevents, or at least mitigate, waste collection blow-outs and leakage. The present invention has a central hole, which is removed to accommodate the ostomy appliance where it attaches to the skin barrier/wafer and this more securely adheres the ostomy appliance and skin barrier/wafer to the patient's skin.

The present invention has many uses, especially in the fields of medicine and medical devices. It will function as an accessory to an ostomy or other appliance, or may be manufactured as a component piece of each appliance. In a preferred embodiment, the present invention will feature a lightweight, pressure-sensitive adhesive, such as Tegaderm™.

The present invention will be helpful to patients with all forms of urostomies, colostomies and ileostomies, including, but not limited to, temporary colostomy, permanent, colostomy, sigmoid or descending colostomy, transverse colostomy, loop colostomy, and ascending colostomy. It can be used with one-piece and two-piece colostomy and ileostomy pouch systems, as well as with irrigation and other systems which often require a cap or other protective device over the stoma.

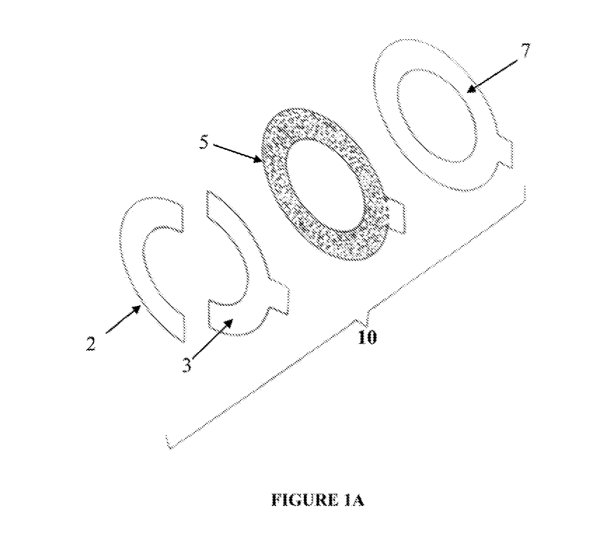

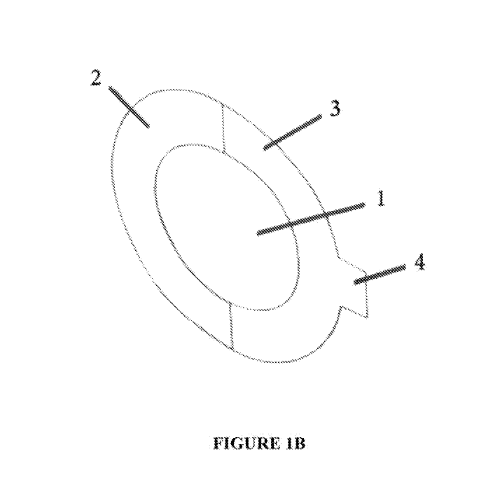

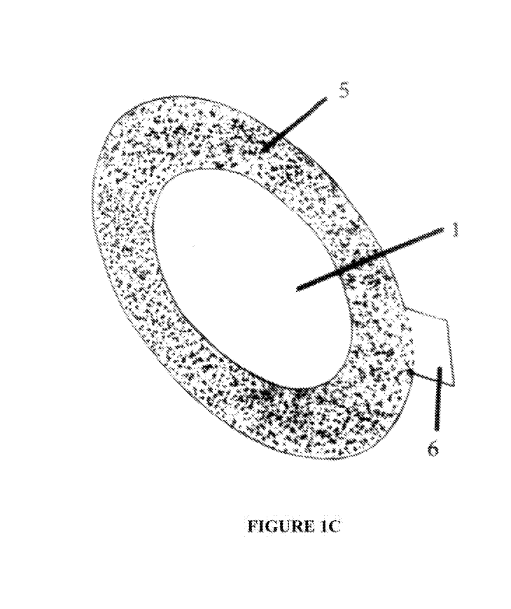

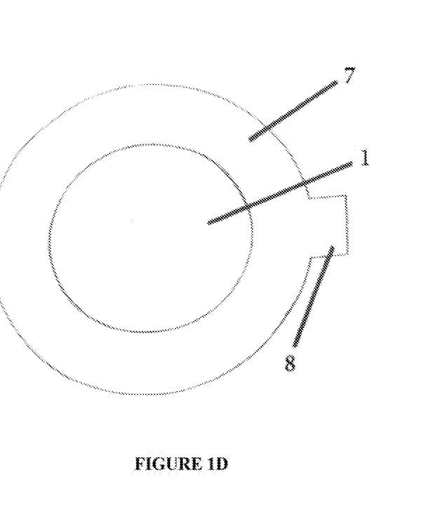

A more detailed description of the present invention of the ostomy appliance collar is as follows and incorporates the following primary components: an interior cut-out section or opening—1; at least two separate arched sections of release liner—2, 3 which will be proximal in relation to the user's body prior to their removal; release liner tab—4; intermediate adhesive layer—5; adhesive layer perforated tab—6; distal support layer—7; and support layer tab—8. The collar, shown in FIGS. 1A-1D can be made from a very thin, soft, pliable, plastic, transparent, pressure-sensitive, adhesive sheet material made for long-term and continuous use on the skin. FIG. 1A shows an exploded view of the component layers of the intact collar 10, further illustrated in FIGS. 1B, 1C, and 1D. FIG. 1B shows the back view of the collar with the center cut-out section 1 and the release liner 2, 3 attached in two separate arched sections, with the release liner tab 4 on the release liner 3. In. FIG. 1C, the intermediate adhesive layer 5, with the adhesive layer perforated tab 6 is shown, with its relation to the center cut-out section 1. As seen in FIG. 1D, the front face of the collar is covered by a support layer 7 with a support layer tab 8 and the center cut-out section 1.

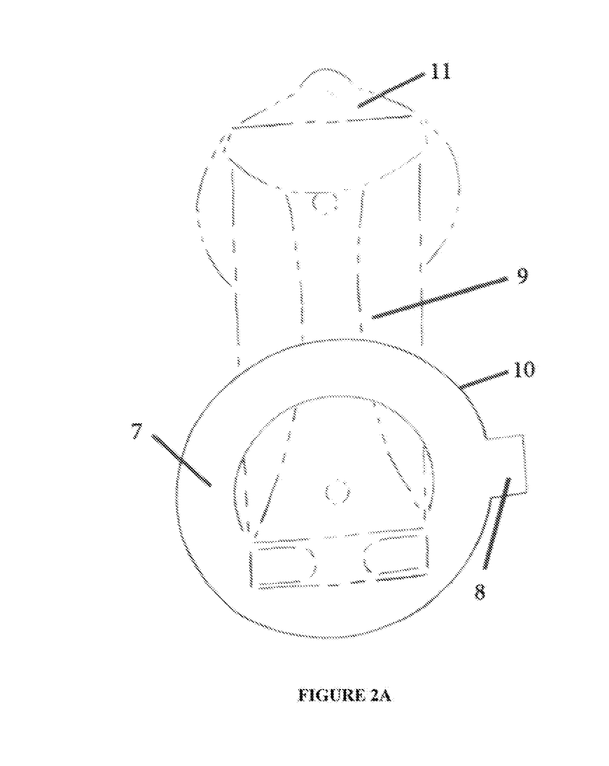

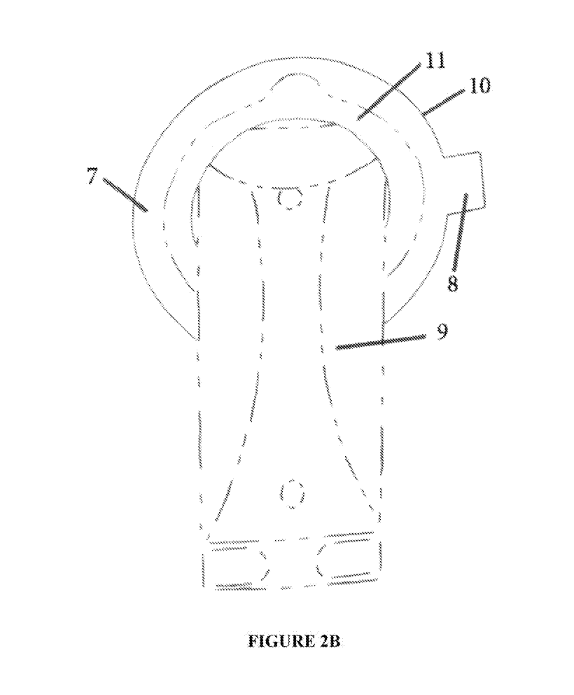

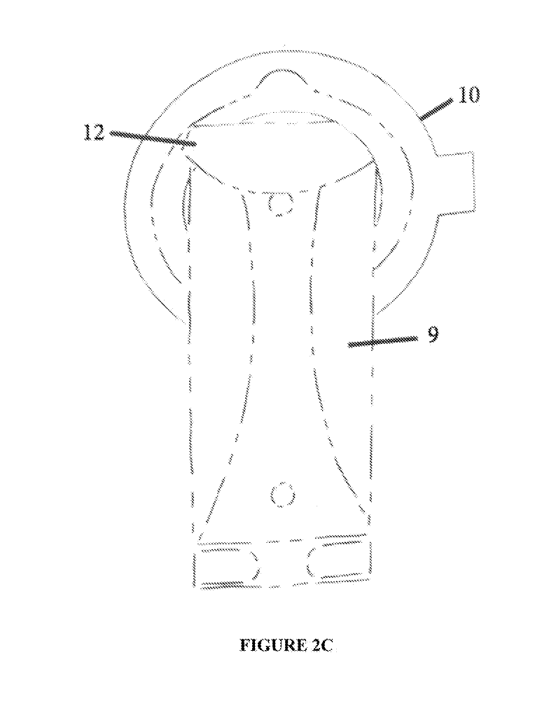

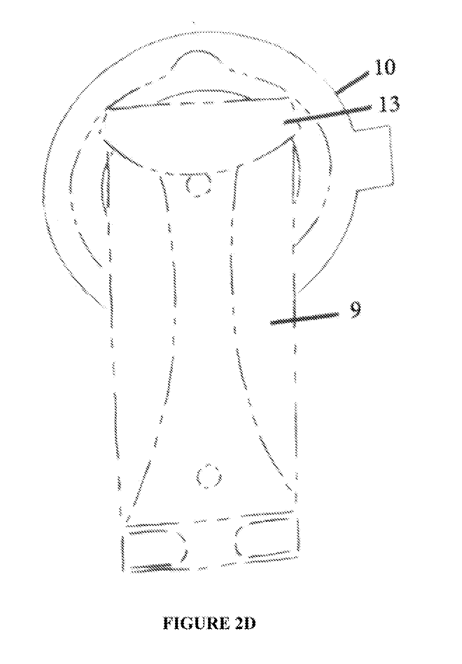

In order to understand how the invention works, it is necessary to refer to the drawings in FIGS. 2A-2D. In FIG. 2A, the ostomy appliance 9 is folded or otherwise reduced in size at the point of collar application and the collar 10 is placed at the detached bottom of the folded ostomy appliance 9 with the collar's support layer 7 and the support layer tab 8 facing away from the ostomy appliance 9 and the body of the user. The ostomy appliance 9 is then maneuvered (by pushing and/or pulling) through or about the collar 10, beginning with the bottom, detached portion of the appliance and continuing to draw the collar toward the body of the user, as shown in FIG. 2A to ultimately finish at the fixed point of contact of the appliance and the user either the skin barrier or a wafer 11. FIG. 2B illustrates the direction, movement, and placement of the collar 10 with the support layer 7 and support layer tab 8 facing forward as it is moved up the folded ostomy appliance 9 to the skin barrier/wafer 11 area. FIG. 2C shows the folded ostomy appliance 9 with the upper left corner portion of the appliance 12 gently maneuvered through the collar 10. FIG. 2B shows the folded ostomy appliance 9 with the upper right corner of the appliance 13 gently maneuvered through the intact collar 10. The collar is now properly positioned to begin the attachment process.

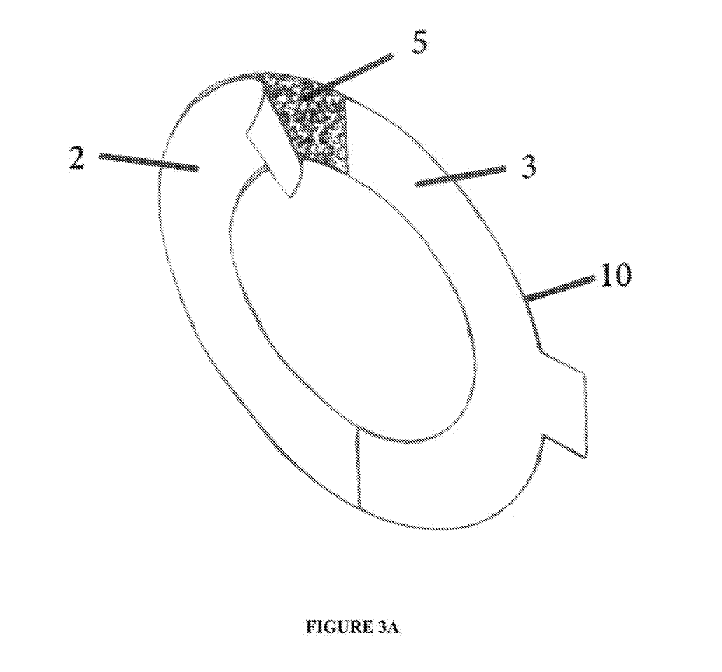

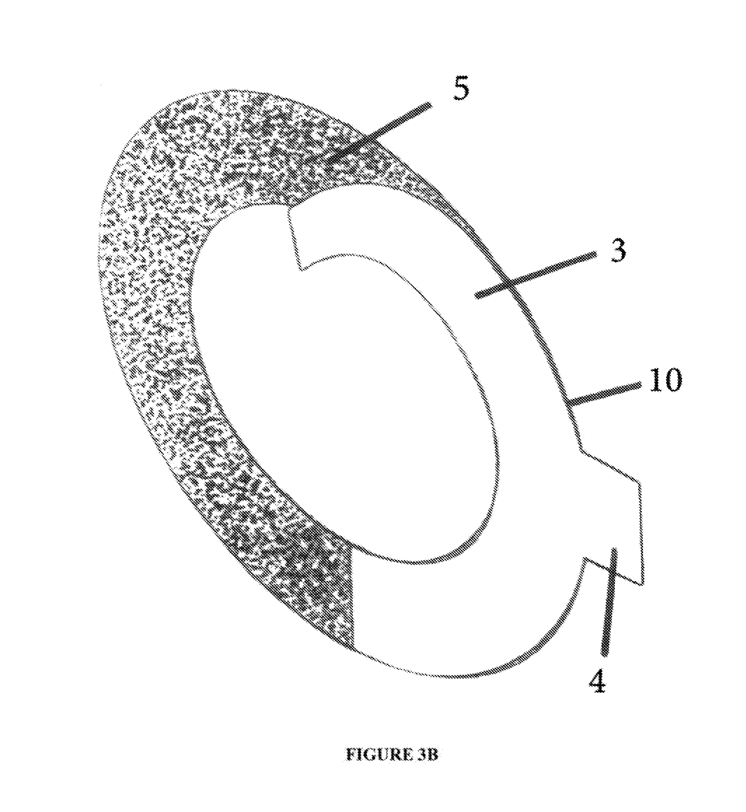

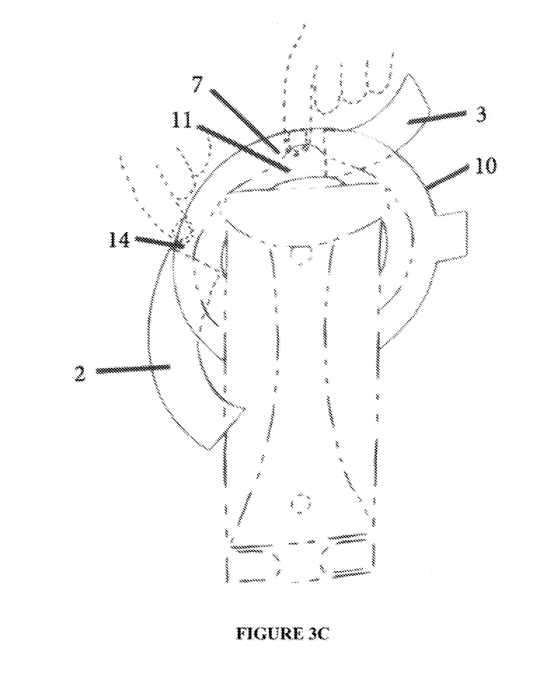

FIG. 3A shows the order in which each release liner 2, 3 is removed from the adhesive layer 5 of the collar 10. FIG. 3B shows the release liner 3 with the release liner tab 4 being removed from the adhesive layer 5 of the collar. FIG. 3C demonstrates how each side of the release liner 2, 3 is removed separately while gentle pressure is applied to the support layer 7 of the collar. This gentle pressure slides along the collar, on top of the support layer 7, slightly behind and in the same direction as the stripping of the release liner 2, 3. After both sides of the release liner 2, 3 have been removed, firmer pressure is applied to secure and seal the collar, which is now minus the release liners 16, to the front of the skin barrier/wafer 11 and the user's peristomal beyond the perimeter of the skin barrier/wafer 14.

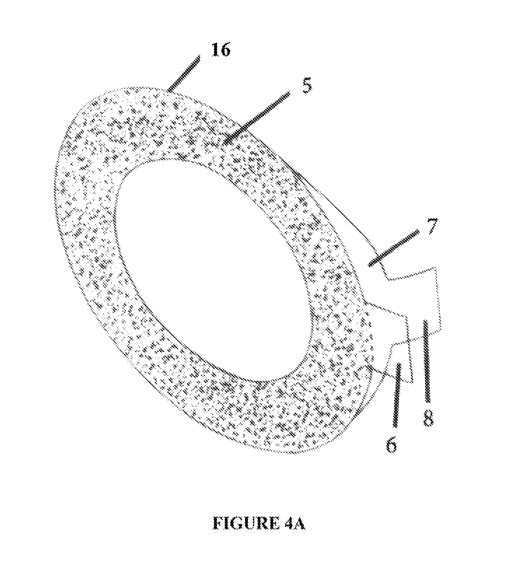

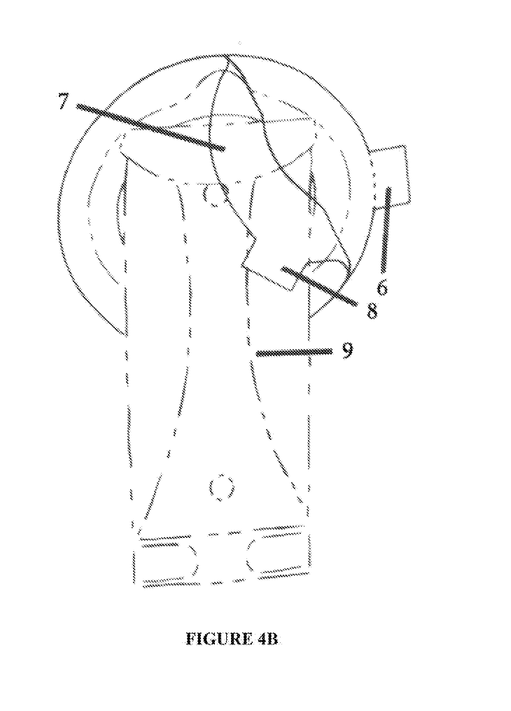

FIG. 4A shows the adhesive layer 5 of the collar 16 with the support layer 7 and support layer tab 8 being removed from the hacking layer with the adhesive layer perforated tab 6. The adhesive layer 5 is on the opposing side of the backing layer with the adhesive layer perforated tab 6. FIG. 4B illustrates how to remove the support layer 7 by gently stripping the support layer 7 along with the support layer tab 8 off of the backing layer with the adhesive layer perforated tab 6 and moving the support layer 7 over, down, and off of the folded ostomy appliance 9. The next step is to hold down the backing layer with the adhesive layer perforated tab 6 near the perforation and remove the adhesive layer perforated tab 6.

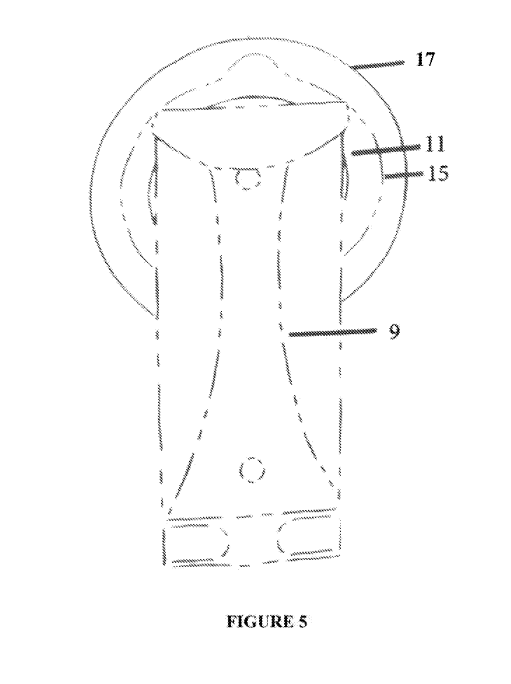

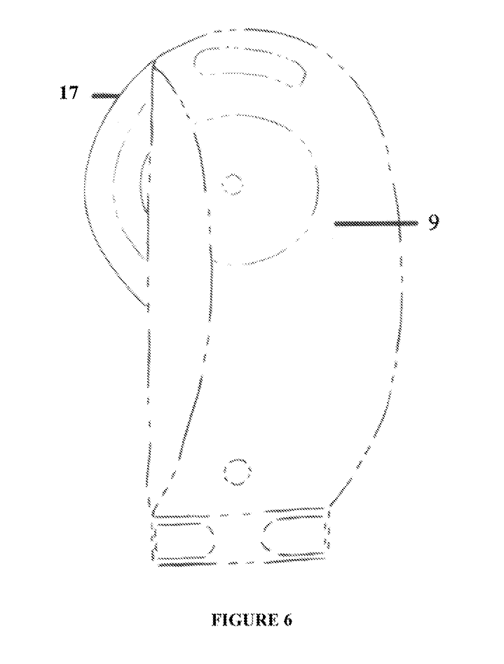

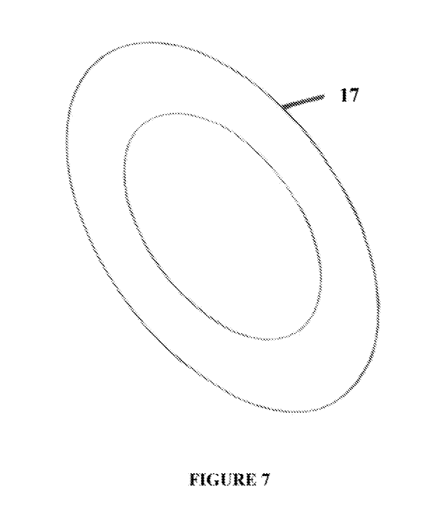

FIG. 5 completes the ostomy appliance 9 and attached collar 17 showing the significant overlap of the collar 17 from skin barrier/wafer 11 to the user's, skin beyond the perimeter of the peristomal skin 15. FIG. 6 shows the collar 17 and ostomy appliance 9 unfolding to the applied, in-use position. FIG. 7 shows the potentially transparent and discreet nature of the fully applied collar 17.

The shapes of the skin barrier/wafers of different manufacturers vary greatly and can be circular, rectangular, square, elliptical, oval, etc. The present invention contemplates numerous possible shapes, which may correspond to the various perimeter sizes, circumferences and shapes of these skin barrier/wafers. The inside diameter of the invention depends strictly upon the shape of the connection point of the skin barrier/wafer to the pouch. Most connection points are circular; however, some are oval and need to be measured and sized accordingly. For purposes of this description, the manufacturer's skin barrier/wafer is thought to be oval and has been measured and sized to 3½ inches. Usually these connection points are circular and have a diameter which can vary from 1¼ inches to 3½ inches. The outside perimeter of the invention will also vary due to, as stated above, the variety of skin barrier/wafers produced by different manufacturers. However, in reference to the outside perimeter of the present invention, the overlap of collar from skin barrier/wafer perimeter to skin area can be, in a preferred embodiment, approximately one inch. The configuration of the invention is key to reducing and, in most cases, catching leaks before they cause skin problems or soil clothes, etc. In addition, the overlap of the collar from the perimeter of the skin barrier/wafer to the skin is essential to covering the perimeter edge which gets caught on clothing during normal activity and can cause the skin barrier/wafer to start to rub and peel away from the body causing leakage. The present invention is created as a one piece unit which provides added support to the ostomy appliance by absorbing pressure exerted from the skin barrier/wafer or the filling ostomy bag. The mechanism of this pressure absorption is as follows: the skin barrier/wafer has adhesive on the body-facing side of the skin barrier/wafer only; during routine use, this adhesive can be eroded and break down; the present invention—positioned over the skin barrier/wafer—would provide the adhesion necessary to stabilize and support the entire appliance/apparatus and the pressure exerted would be absorbed evenly by the present invention due to its circular, continuous configuration. The present invention can be employed in conjunction with an ostomy appliance, in the normal and regular course of use, manufactured as part of the ostomy appliance, or it can be carried along by the user to resolve or mitigate a problematic situation, in the event of a leak or other highly personal and traumatic occurrence.

### MATERIALS AND METHODS

Applicant has built a prototype of the present invention, and considered and experimented with multiple possible configurations and various materials for putative manufacture.

We claim:

1. An ostomy appliance collar for use in conjunction with an ostomy appliance comprising the following component layers, each with an interior cut-out section:
(a) two or more separate arched sections of release liner; and
(b) an adhesive layer; and
(c) support layer.

2. The collar of claim 1 wherein the two or more separate arched sections of release liner and the support layer are removably affixed to either side of the adhesive layer.

3. The collar of claim 1 wherein the interior cut-out section is circular in shape and centrally located within each layer.

4. The collar of claim 1 further comprising:
(a) a release liner tab;
(b) an adhesive layer perforated tab; and
(c) a support layer tab.

5. The collar of claim 1 which is made of thin, soft, pliable, transparent, pressure-sensitive, materials with selectively adhesive properties.

6. The collar of claim 1 which is utilized in conjunction with an ostomy appliance and a sealing wafer.

7. The collar of claim 1 which is utilized in conjunction with an ostomy appliance in place of a sealing wafer.

8. A method to reduce leakage of waste from the receptacle device of an ostomy appliance comprising:
(a) positioning the collar of claim 1 such that the two separate arched sections of release liner are in proximal relation to the ostomy appliance; followed by the intermediate adhesive layer; and distal support layer;
(b) placing the collar at the detached lower portion of the ostomy appliance, which can be folded or otherwise manipulated to accommodate the collar, if necessary;
(c) drawing the collar around and about the ostomy appliance by way of its interior cut-out section until the collar reaches the fixed point of attachment of the ostomy device to the skin of the user or ostomy device or other accessory;
(d) separately removing each section of the release liner;
(e) securing the collar to the skin of other surface; and
(f) removing the support layer by moving it over, down, and off of the ostomy appliance.

9. The method of claim 8 further comprising:
(a) separately removing each section of the release liner by applying gentle pressure along the collar, on top of the support layer, slightly behind and in the same direction as the stripping of each section of the release liner;
(b) applying firmer pressure along the collar to secure and seal the collar to the skin or other surface;
(c) removing the support layer by gently stripping the support layer along with the support layer tab off of the backing layer with the adhesive layer perforated tab and moving the support layer over, down, and off of the ostomy appliance;
(d) removing the adhesive layer perforated tab by holding down the backing layer near the perforation and pulling off the adhesive layer perforated tab.

10. A one-piece ostomy appliance for receiving discharge from a stoma of a user-patient, comprising:
(a) a receptacle device;
(b) a skin barrier or other interface for attachment to the skin of the user; and
(c) the collar of claim 1 which is loosely attached to the appliance in proximity to the skin barrier and which can be applied by the user by removing each layer.
